(19) United States
(12) Patent Application Publication
(10) Pub. No.: US 2018/0147124 A1
(43) Pub. Date: May 31, 2018

(54) NON-INTRUSIVE BREAST MILK MONITORING

(71) Applicant: Leo Adam Drew, San Antonio, TX (US)

(72) Inventor: Leo Adam Drew, San Antonio, TX (US)

(73) Assignee: THE BOARD OF REGENTS OF THE UNIVERSITY OF TEXAS SYSTEM, Austin, TX (US)

(21) Appl. No.: 15/826,220

(22) Filed: Nov. 29, 2017

Related U.S. Application Data
(60) Provisional application No. 62/427,595, filed on Nov. 29, 2016.

(51) Int. Cl. A61J 15/00; A61B 5/00; A61J 11/00

(52) U.S. Cl. CPC A61J 15/0084; A61B 5/4288; A61B 5/6823; A61B 5/6833; A61J 11/0005; G06F 19/3406; A61B 2503/045; A61B 2503/12; A61B 2560/0214; A61B 2560/0462; A61B 5/742

(57) ABSTRACT

Certain embodiments are directed to methods, apparatuses, and systems for providing accurate, non-intrusive measurement of breastfeeding. The embodiments described herein are used during the normal breastfeeding routine and measure the volume of breast milk transferred in real time. The device is lightweight, disposable, and does not interfere with the milking mechanisms used by infants. The device provides a more accurate monitoring of infant nutrition and peace of mind for mothers.

### CROSS REFERENCE TO RELATED APPLICATIONS

The present application claims priority to U.S. Provisional Application No. 62/427,595 filed Nov. 29, 2016, which is incorporated here by reference in its entirety.

### STATEMENT REGARDING FEDERALLY FUNDED RESEARCH

None.

### BACKGROUND OF THE INVENTION

### Field of the Invention

The application relates to systems, apparatuses, and methods for non-intrusively monitoring breast milk flow from a breast to an infant.

### Description of Related Art

When a new mother prepares to leave the hospital with her infant after childbirth, her primary concern is the proper nutrition of her baby. The current method of gauging infant nutrition calls for inspection of the infant's diapers for signs of urination and defecation. Prior to birth, infants gain a considerable amount of water weight to help them squeeze through the birth canal. That water weight is later expunged from the body via urine and excrement. This creates inaccuracies and inconsistencies with the visual inspection of an infant's intake. It renders the traditional method inadequate and creates a need for a device that measures the volume transferred from mother to infant in real time.

Infant malnutrition occurs in 10% of infants in the US alone, and over 50% of mothers surveyed feel unsure about the amount of milk their infant receives through breastfeeding. The inaccuracy of current measurement methods stems from the disruption of the typical breastfeeding process and the tendency of a baby to expel bodily fluids unexpectedly.

### SUMMARY OF THE INVENTION

The present disclosure includes embodiments of methods, apparatuses, and systems for providing accurate, non-intrusive measurement of breastfeeding while maintaining the important bond between mother and child. The embodiments described herein are used during the normal breastfeeding routine and measure the volume of breast milk transferred in real time. The device is lightweight, disposable, and does not interfere with the milking mechanisms used by infants. The device provides a more accurate monitoring of infant nutrition and a peace of mind for mothers.

The non-intrusive breast milk monitor described herein can accurately measure the volume of breast milk transferred to a human infant during breastfeeding. The device is designed to minimize interference between mother and child.

In one aspect, the systems and techniques disclosed here feature a breast milk monitoring device including a top surface and bottom surface, where the bottom surface is placed against a woman's breast. For example, the bottom surface may be a polymer adhesive patch that is placed over and secures around a woman's nipple. In certain aspects there is adhesive in one or more concentric circles along the underside or bottom surface of the patch that secures the device to the breast. In certain aspects when the device is in use the adhesive circumscribes the nipple and removeably adheres the device to the breast. A portion of the device is configured to be positioned over the nipple, and is positioned internal to the adhesive, forms a cavity to allow for milk collection, forming a breast milk collection portion. In certain aspects the breast milk collection portion is void of adhesive. The material used for the breast milk collection portion is thin enough to allow for peristalsis of the infants tongue to extract the milk from the nipple when the device is in use. Once the milk is in the cavity, gravity and/or negative pressure carries the milk through an inlet channel (e.g., tubing) to a sensor where the milk enters the sensor through a sensor inlet. In certain aspects the device includes a self-contained power source or battery to power the sensor. The milk flows through the sensor and to a sensor outlet. The sensor outlet is fluidly connected to an outlet channel (e.g., tubing) that leads to a faux nipple or infant feeding portion. The milk flows from the sensor to the faux nipple by way of negative pressure created from the infant's suckling action. In certain aspects the infant feeding portion is positioned on top of the milk collection portion. The breast milk flow path leads from the milk collection portion, flowing through the sensor to the faux nipple. In certain aspects the faux nipple is located on the top center of the device, where the milk is accessed by an infant via suckling.

The device can be used to accurately measure the amount of milk that an infant imbibes while breast feeding, without interfering with the peristalsis. The amount of milk measured is indicative of the amount of milk that would be transferred without the device present.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 is a top view of one embodiment of the breast milk monitoring device.

FIG. 2 is a close-up perspective view of one embodiment the breast milk collection portion.

FIG. 3 is a side view of one embodiment the breast milk monitoring device.

FIG. 4 is a cross-sectional view of one embodiment the breast milk monitoring device.

### DETAILED DESCRIPTION OF THE INVENTION

The below description includes several embodiments of the present system and methods. While exemplary systems are described, various modifications will be apparent.

The breast milk monitoring device will measure the volume of milk transfer during a normal breastfeeding session. The device will be non-intrusive and allow for the feeding to occur in a manner similar to feeding when the device is not present. The device is intended for periodic use by lactating mothers concerned about the amount of milk imbibed by their infant. It will also have applications with lactation consultants to aid with the habilitation of feeding as well as with healthy newborns, NICU, late preterm, down syndrome, and babies with palate deformities.

The device can be comprised of a polymer that is thin enough to allow for an unhindered experience, while still being strong enough to withstand the forces and pressures inflicted on it. The polymer will be secured to the breast around the exterior perimeter of the areola and will create a cavity over the nipple. There will be a port with a channel or tube attached to the cavity to allow the milk to be transmitted to a sensor. The sensor will accurately measure the volume of breast milk transferred to a baby during breastfeeding in real-time. The milk will be transmitted to a faux nipple to be relayed to the infant for ingestion. The infant's mouth should be placed as usual according to the mother/infant's preference. The volume data can be transferred from the sensor to a USB drive or directly to a computer containing software that is complementary to the sensor. The USB drive can relay the data to the computer and clearly display it in the software.

### I. System Embodiment

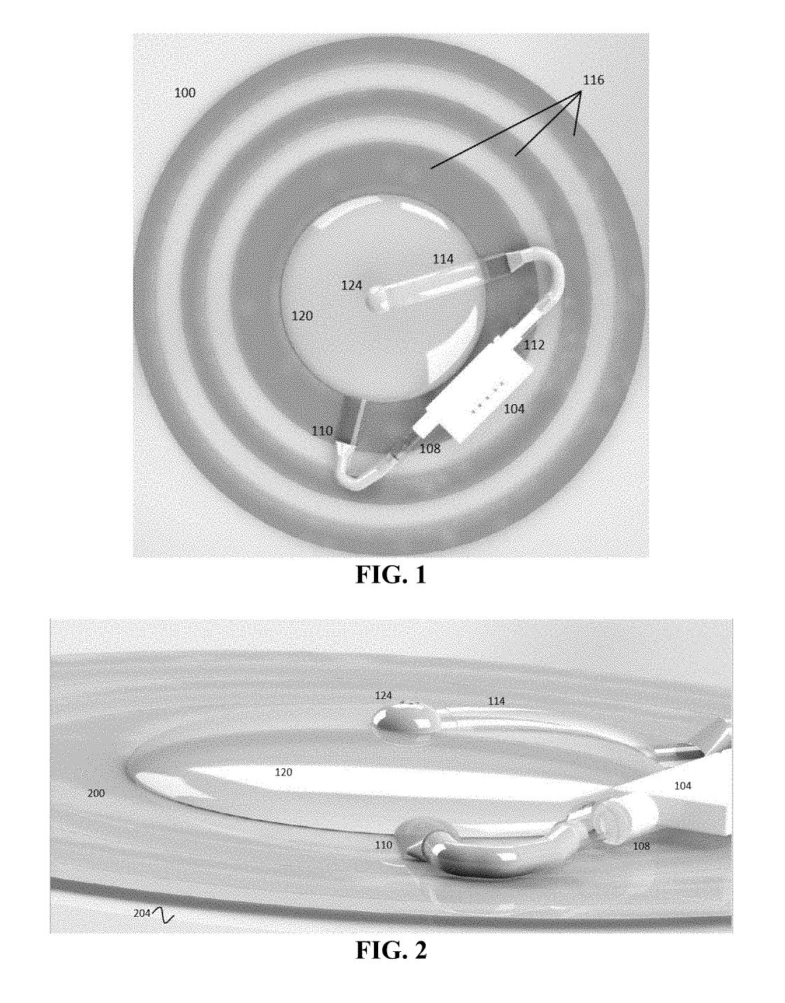

Referring to FIGS. 1-4, breast milk monitoring device 100 includes bottom surface 204 that is placed against a subject's breast and top surface 200. Attachment portion 116 fully or partially circumscribes subject's nipple and removeably adheres device 100 to the subject's breast. Attachment portion 116 may include an adhesive bottom surface 204 in the shape of one or more concentric rings. These concentric adhesive rings can be designed to accommodate variety of areola sizes.

A breast milk collection portion 120 forms a cavity over the subject's areola when the device 100 is attached to the subject's breast. Device 100 also includes a sensor 104 having an inlet 108 and outlet 112. The sensor is intended for use in an indoor, low-moisture environment. Inlet 108 is connected to the collection portion 120 by an inlet channel 110. Breast milk flows from the collection portion 120 into the inlet 108 through inlet channel 110 by, for example, way of gravity.

Device 100 also includes a faux nipple 124 to simulate a mother's actual nipple to encourage suckling. Faux nipple 124 is connected to the sensor outlet 112 by an outlet channel 114. Breast milk flows from sensor outlet 112 through outlet channel 114 to the faux nipple 124 by way of negative pressure created from the infant's suckling action.

In a particular implementation, the smallest adhesive concentric ring of the attachment portion 116 is at least 5 inches in diameter and may include a thin polymer membrane coated with a layer of adhesive (e.g., Tegaderm 3M) to create a watertight seal. Sensor 104 (e.g., the LD20 or Sensirion® SLQ-QT500 liquid flow sensor by Sensirion®) is configured to measure flow between approximately 1 ml/min and 10 ml/min. In certain aspects the sensor can be powered by a battery and/or a powered connection (e.g., a USB connection). The powered connection can also be capable of transferring data (directly or wirelessly) to a computer for display. In certain aspects the sensor will measure volume within ±0.15 mL/min of the full scale flow. To ensure minimal interference, the sensor can be less than approximately 2.4 inches long and 1.4 inches wide. The faux nipple 124 can be made from a malleable polymer such that the polymer will allow peristalsis suitable for milk expression. The polymer material should allow the nipple and areola to form a teat inside the infant's oral cavity. Inlet channel 110 and outlet channel 114 may be formed from medical grade tubing such as a sturdy, transparent plastic polymer. In certain aspects the tubing, cavity and channels can be transparent to allow for viewing. Externally, the channel or tubing will not be exposed to any extreme conditions. Internally, it will experience pressure forces from the fluid's motion. The total length from the inlet channel 110 to the outlet channel 114 should not exceed 12 inches, with the total length of tubing used for the inlet channel 110 and outlet channel 114 not to exceed 4 inches. The entire device 100 should weigh no more than one pound. The device will typically be operated at room temperature (68° F.-72° F.).

Other implementations are contemplated. For example, sensor 104 may optionally be configured to measure caloric content of the breast milk or the strength of an infant's suckle. Sensor 104 may also be configured to display volumetric flow and total output volume of breast milk. Moreover, any combination of collection portion 120, inlet 108, inlet channel 110, outlet 112, outlet channel 114, or faux nipple 124 may be configured to receive medicine or other supplements. The faux nipple 124 may optionally be configured to indicate how far the nipple is inserted into the infant's mouth. In other implementations, sensor 104 may include a memory to store a plurality of data elements. A processor may execute instructions to the memory to store data elements and send the data elements to a user device for graphical display.

While not in use, the device may be freely transported to other locations via land, air, or sea. The device is intended for use at home or in a lactation room. In certain aspects the device can be used during a routine feeding (10-30 minutes) and can be disposed of afterwards.

The software is intended for maintenance-free operation. If a need to replace software arises, a USB can be repurchased.

The packaging must be sterile, tear resistant, breathable with a microbial barrier. The packaging will be compatible with the following sterilization methods: ethylene oxide (EO), gamma, electron-beam, steam under controlled conditions and low temperature oxidative sterilization.

This design is intended to allow breastfeeding to occur unimpeded while obtaining accurate volumetric flow measurements. Its soft polymer design allows an infant to naturally express milk from its mother's breast via peristalsis. This expressed milk is trapped inside a cavity formed by a ring of adhesive that creates an airtight seal around the nipple. An adhesive ring extend radially outward on the underside of the patch and ensures it is properly secured. This adhesive ring accommodates a variety of areola sizes. Once trapped in the cavity, the milk flows through a channel, formed by adhesive, toward the outer radius of the patch. Just a short distance outside of the initial cavity, the milk is transferred to the top side of the patch and into the LD20 sensor via medical grade tubing. It is then transferred back toward the center of the patch where it will transition from medical grade tubing back to a soft, polymer channel. This channel will direct the breastmilk to the center of the patch where it will enter the infant's mouth through a small, faux nipple.

In certain aspects a sensor has a maximum sensing flow rate of 10 mL/min and a minimum sensing flow rate of 0.10 mL/min. The sensor is disposable and designed for biomedical applications. It also permits and measures bi-directional flow, which may be important in the case of backwash during breastfeeding. It is powered via USB cable that also allows for the transfer of real-time data to an incorporated software allowing for quick visualization and interpretation of the data. The overall dimension of the sensor can be 0.1, 0.25, 0.5, 0.75 to 1.0, 1.25, 1.75, 2.0, 3.0 inch (including all values and ranges there between)×0.1, 0.2, 0.3, 0.4, 0.5 to 0.6, 0.7, 0.8, 0.9, 1.0, 2.0 inch (including all values and ranges there between)×0.1, 0.2, 0.3, 0.4, 0.5 to 0.6, 0.7, 0.8, 0.9, 1.0, 2.0 inch (including all values and ranges there between). In certain aspects the overall dimensions of the sensor are 1.77 in.×0.55 in.×0.47 in and it weighs approximately 2.1 grams. Other flow sensor can be substituted for the LD20.

In other aspects the sensor has a liquid flow rate of up to 120 ml/min and as low as 0.15 ml/min. This sensor is suited to a wide range of viscosities and has a response time of less than 50 ms. The sensor can be powered by a USB cable which can also transfer data to a computer and comes complete with software to display and interpret the data or battery powered. It can also have the capability for an analog output with of voltage of between 0 and 10 V. The sensor can have an overall dimensions of 2.32 in×1.38 in×0.91 in and weighs 45 g. The tubing can be PFA ¼″ for the connection.

The tubing that leads from the sensor is then directed back to the surface of the polymer patch. On the surface of the patch, the tubing will meet and connect with another narrow layer of polymer that creates a channel directing the flow of milk toward the center of the patch. At the center of the patch a faux nipple is located, and it is here that the channel is connected. In the faux nipple, the milk is directed up holes in the tip and to the infant's mouth.

Alternative designs were created with larger volumes in mind. The Sensirion® SLQ-QT500 sensor or similar sensors, are accurate for small quantities as well as large quantities and can handle a large volume feeding, greater than any single feeding could ever produce. The integrated USB allows for the device to be powered and transmit data with one cable. This sensor comes with medical tubing already attached to both the inlet and outlet making it one less connection open to failure. The sensor is designed considering biological fluid and is therefore safe to use by mother and infant.

A smaller sized sensor will take up less space on the breast and be less bulky. For this application, two sensors are considered as examples. The first, LD20, is 1.78 in long, 0.56 in wide, and 0.48 in tall. It has a total volume of 0.478 in³. The second, SLQ-QT500, is 14.17 in long, 1.4 in wide, and 0.92 in tall. It has a total volume of 18.251 in³. Small sensor size is not vital but preferred. It is weighted at 2 points.

Certain embodiments include a short flow path (between 0.1 to 5 cm, including all values and ranges there between), one adhesive ring, and a lightweight sensor capable of measuring the necessary flow range.

The adhesion strength of materials is affected by the structure, surface energy, and viscoelastic properties of the skin on which it is applied. Tegaderm 3M, a transparent film dressing, is commonly placed on the breast and areola after mastopexy. It has a low removal force on skin (0.96 N for a 1.4 in2 area) and low pain intensity. Other low trauma adhesives have an average peel force of 3.78 N.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A Representative Non-Intrusive Milk Monitoring Device

Sensor.

The LD20 sensor in one example of a sensor that can be used. The LD20 has a maximum sensing flow rate of 10 mL/min and a minimum sensing flow rate of 0.10 mL/min. Its capability is well within the range of normal breastfeeding flow rates, but may fall short in rarer cases of high milk production. The sensor is disposable and designed for biomedical applications. It also permits and measures bi-directional flow, which may be important in the case of backwash during breastfeeding. It is powered via USB cable that also allows for the transfer of real-time data to an incorporated software allowing for quick visualization and interpretation of the data. The overall dimensions of the sensor are 1.77 in.×0.55 in.×0.47 in and it weighs approximately 2.1 grams. The sensor also has luer lock connectors at its inlet and outlet, allowing for easy securement of tubing to the sensor.

Tubing.

An example of a tubing material is Tygon 2375 Ultra Chemical resistant tubing. This tubing provides minimal frictional resistance and contains an inner diameter of 0.102362 in. It has a wall thickness of 0.03125 in. and an outer radius of 0.133612 in. Its minimum bend radius of 0.25 in. provides the desired capability for acute angles in the design. The maximum working pressure for the tubing is 25 psi, exceeding the necessary amount of 6 psi by approximately four times. Most importantly, it is chemically resistant, will not deteriorate, and is in compliance with FDA food contact regulations.

Solidworks Simulations.

Using a solid, curvature based mesh, the infant's suction power was tested against the adhesive's attachment force. A study determining stress, displacement, strain, and factor of safety were executed. The high quality mesh has 28,752 nodes, and 14,052 elements. The minimum element size is 0.0609003 in and the maximum size is 0.301502 in.

Because if the limited library of material properties, low density polyethylene (LDPE) was used instead of polyurethane (PU). LDPE has a maximum operating temperature of 160° F., a tensile strength ranging from 1.8 to 2.2 kpsi, and an elongation percentage of 600%. PU's has a maximum operating temperature of 200° F., a tensile strength ranging from 3.6 to 6.5 kpsi, and an elongation percentage ranging from 465% to 530%. The differences in operating temperature are not relevant because the device will operate at room temperature. PE is much stronger and only slightly less ductile than LDPE, so the results provide a good approximation of real-life outcomes.

The area closest to the infant's mouth experiences the greatest displacement. The highest value, obtained at the faux nipple, is 0.01535 in. Because the material used has a high elongation percentage, this is not a concern. The polymer's properties are adequate for the loading experienced. Other areas, further away from the oral cavity, such as the concentric adhesive ring, experience little to no displacement.

Overall, LDPE handles the applied forces well. The most prominent areas of mid-range stress are at the material joints and around the faux nipple. The stress never reaches above 244.5 psi and PU can withstand more.

Because strain is a ratio of change in length over length, it is unitless. Once again, the major areas of stress concentration are the material joints and around the faux nipple. The maximum strain experienced was roughly 0.008477. Despite the applied infant suction, the patch does not deform significantly.

Overall, the adhesive patch has a factor of safety of 2.726. Its material and design properties exceed those required for safe operation at the infant's maximum suction pressure.

In certain aspects the total length of the flow path from the mother's nipple to the baby's mouth is calculated to be 7.505 in. The Darcy-Weisbach equation is used to determine the head and pressure loss through the total flow path. The Reynold's number and Darcy Friction Factor must first be calculated. A constant diameter of 0.102362 in. and a fluid velocity of 0.741535 in./s are applied. The fluid velocity is calculated from the volumetric flow value of 6 mL/min and the tubing diameter. This volumetric flow value is calculated from the maximum average volume of breast milk in a feeding divided by the minimum time for a breastfeeding session. This provides the highest typical flow value, causing the greatest pressure drop and validating all lower flow values.

$$Re = \frac{ρ VD}{μ} = \frac{( 16.895 g \text{/} in {.}^{3} ) ( 0.741535 \frac{in .}{s} ) ( 0.102362 in . )}{( \frac{0.034544 g}{in . * s} )} = 37.12$$

The flow is laminar according to the Reynold's number. Therefore, the friction factor is calculated using the equation below.

$$f = \frac{64}{Re} = \frac{64}{37.12} = 1.724$$

These values are now applied to calculate the head loss in the flow path. The minor losses for the inlet, outlet, and bends in the tubing are accounted for. 1, 0.5, 0.2, and 0.2 are the values applied for these losses, respectively. The inlet and outlet conditions are assumed to be unshaped, sharp corners and for simplicity each bend is approximated to be 180 degrees. This should offer a slight overestimation of the actual minor losses.

$${h}_{L} = \frac{Δ {P}_{L}}{ρ g} = ( f \frac{L}{D} + {K}_{L} ) * \frac{{V}_{avg}^{2}}{2 g} = ( 1.724 * \frac{7.505 in .}{0.102362 in .} + 1.9 ) * \frac{{( 0.741535 \frac{in}{s} )}^{2}}{2 * ( 32.2 \frac{ft}{{s}^{2}} ) * ( \frac{12 in}{ft} )} = 0.091 in . \\ Δ {P}_{L} = 0.0034 psi$$

Typical infant sucking pressure ranges between 4 and 6 psi. The calculated pressure loss of 0.0034 psi is easily overcome by the capability of the infant's applied negative pressure.

The primary dimensions of the patch were determined through research of the average areola size with a 3σ variance. This research determined the required size for the areola cavity. The maximum diameter of the patch was then designed to allow for a reasonable surface area of adhesive to ensure that the patch remains securely attached during use. The maximum flow path and tubing diameter were determined to minimize head loss, fluid entrapment, and hydraulic resistance.

Test Methodology for Electrical/Electronic Systems.

The LD20 liquid flow sensor was purchased from the manufacturer Sensirion®. The sensor included an evaluation kit that came with 3 sensors, PC software, and a SCC1-USB power cable. The sensor used is medical grade, biocompatible, and water resistant. Repeated exposure to small amounts of fluid, imitating infant salivation, tested the system's compatibility for use in the intended environment.

Test Methodology for Control Systems.

A fake breast was created and a negative mold developed out of a 3D printed PLA. A ballistics gel breast was then created from this negative mold. An appropriate length of tubing was inserted into the back of the mold and ends just short of the ballistics gel nipple. A slit was made at the nipple to resemble realistic breast milk flow. A syringe pump was attached to the other end of the tubing and set to a flow rate of 6 mL/min. The pump was filled with warm water to flow through the system. A Breast Intentions patch was then applied as intended over the ballistics gel breast. This test determined whether the sensor and DAQ are accurately measuring and recording the flow of breast milk through the device by comparing the recorded values with the known flow rate.

Test Methodology for Mechanical Systems/Components.

The strength and adhesion of the patch were tested under the conditions of maximum infant suction pressure. The patch was placed on a ballistics gel mold of the breast. A hand vacuum pump was applied to the faux nipple of the patch to simulate the maximum suction pressure of 6 psi.

Test Methodology for Fluid/Pneumatic Systems.

The tubing and adhesive used to secure it was tested as a separate unit. To ensure the tubing system can withstand the maximum expected volume and flow, it was operated at larger fluid quantities and under greater suction pressure. A flow rate of 6 mL/min was pumped through the tubing to properly simulate the breastfeeding experience. The tubing was visually inspected to ensure smooth flow and no leakage.

Test Methodology for Software.

Sensirion® Software is included with each USB. To ensure the data collected and displayed was accurate, the sensor and its accompanying software was tested separately from the collective unit. Predetermined amounts of milk/fluid were inserted into tubing and allowed to flow through the sensor. The data was recorded to confirm accuracy.

Test Methodology for Other.

One aspect of this design is its ability to function in a manner that is non-intrusive to the typical breast feeding process. This is defined by the ability of an infant to utilize peristalsis to express milk from the mother's nipple. The engineers hand and fingers were used to simulate the infants mouth to express the fluid. The patch was again placed over a ballistics gel mold of a breast. The tube running through to the nipple will be attached to a raised reservoir filled with water. The hand and fingers, mimicking peristalsis against the breast. The apparatus will be visually inspected during this process to see if milk is expressed through the nipple.

Software Setup.

In certain embodiments a PC running Windows 7 or later with an available USB port is required. If this is your first time using the device, the sensor requires software to be downloaded on the computer prior to use. To download the software visit sensirion.com/myLMFK on the world wide web. Scroll down to the Software and Drivers section of the page and click the first link, Sensirion® Viewer Software. Once downloaded right click and select run as administrator. Follow the simple prompts on the install wizard to complete the installation. Next click on the link in the same section titled Virtual COM-Port (VCP) Driver. Right click the download and select run as administrator and as before follow the prompts in the installation wizard.

Sensor Setup.

Before using the patch the system must first be set up. To begin the sensor needs to be attached to the computer. To do this the adapter is inserted over the sensor. This can only be done in one configuration and it will fit with ease and minimal force is necessary. The adapter is screwed into the USB cable ensuring that the pins line up correctly with the holes in the USB so they will not be bent. The other end of the USB is then attached to the computer in a standard USB 2 or USB 3 port.

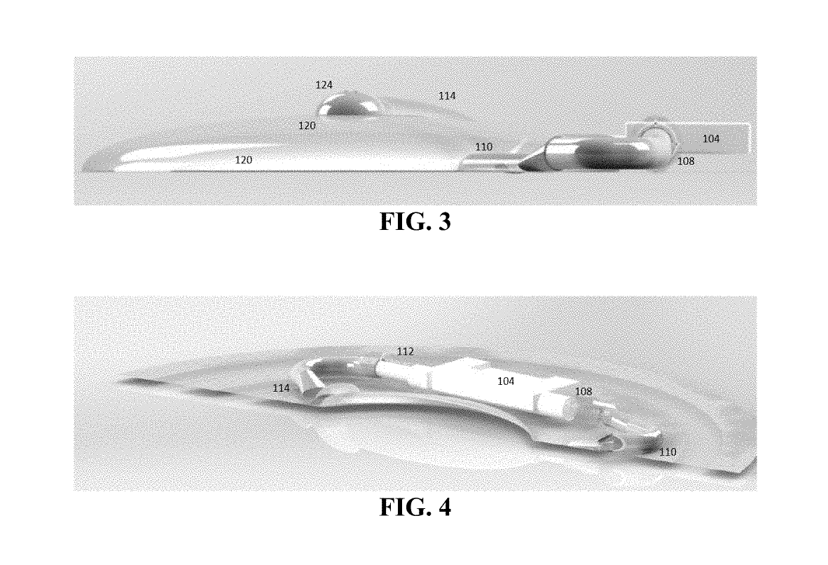

The device will appear in the windows device manager as USB Serial Port. Launch Sensirion® Viewer Software. The Product Selection window will open. Select Liquid Flow Sensor, as seen in FIG. 2, under Sensor Product and RS485/USB Sensor Cable under COM Hardware as seen in FIG. 3. On the right side of the window, the default COM PORT Settings and RS485 Device Settings are recommended. Press OK. The USB/RS485 Sensor Viewer window will appear and indicates a successful connection between the sensor and PC has been established.

Application onto Breast.

Remove excess hair from area surrounding nipple. Sterilize breast tissue using the topical antiseptic wipe provided. Make sure the area is dry and free from oils before proceeding onto the next step. Peel the protective film from the adhesive patch. Ensure nipple is erect. Place the clear center cavity of the adhesive patch over the nipple. When facing the front of the device, the tubing on the left side of the sensor should be placed vertical directly under the areola. Apply pressure to patch while placing it to remove air bubbles.

Infant Setup.

This device is intended for use with mature milk. Place the infant's mouth over the nipple piece. The infant will adjust their hold as needed and may fit more of the teat in their mouth. Once content with placement, the infant will proceed to suckle and use peristalsis to milk the breast. Milk will flow from the inner nipple cavity, through the channels, and to the infant's mouth.

Data Collection.

To begin data collection, click the Select File feature of the software. Once selected, follow by selecting the Run command under Measurement Control to initiate the program. Under the Totalizer tab, click Start to record the quantity of milk transferred in milliliters. At this point the program will be running but will not be logging the collected data. To begin collecting, select the Start Logging button. This will log the information to the desired file location previously selected. The software will display the flow rate in milliliters per hours under Flow Rate.

Sampling time will depend on duration of the feeding session and varies from mother to mother. The Sensirion® software will output sampling time in milliseconds and is displayed at the bottom right corner.

Device Removal.

Once the feeding session is over, remove the infant from the faux nipple. Next click Stop. Clear the values by pressing Reset. Remove the sensor's connection to the USB port. Carefully peel the adhesive patch from the breast tissue. Have a towel on hand to catch any excess fluid that may remain in the patch's channels. Dry the skin. Dispose of the unit.

Data Interpretation Guide.

The amount of milk transferred during breastfeeding varies greatly from mother to mother. Results gathered will need further discussion with your pediatrician to decide proper infant milk intake.

1. A breast milk monitoring device comprising:
a top surface and a bottom surface, the bottom surface configured to be placed against a subject's breast;
a breast milk collection portion that forms a cavity over the subject's areola when the device is attached to the subject's breast;
a sensor having an inlet and an outlet, the inlet being fluidly connected to the breast milk collection portion by an inlet channel;
a faux nipple fluidly connected to the sensor outlet by an outlet channel; and
an attachment portion that fully or partially circumscribes the nipple and removeably adheres the device to the breast.

2. The device of claim 1, wherein the sensor comprises a self-contained power source.

3. The device of claim 1, wherein the attachment portion comprises an adhesive bottom surface of one or more concentric rings extending radially outward.

4. The device of claims 1-3, wherein the sensor is configured to measure caloric content of the breast milk.

5. The device of claims 1-4, wherein the collection portion, outlet, or faux nipple is configured to receive medicine or supplemental nutrition.

6. The device of claims 1-5, wherein the sensor is configured to measure the strength of an infant's suckle

7. The device of claims 1-6, wherein the faux nipple is configured to indicate how far the nipple goes into the infant's mouth.

8. The device of claims 1-7, wherein the sensor is configured to display volumetric flow and total volume.

9. A breast milk monitoring device comprising:
a top surface and a bottom surface, the bottom surface configured to be placed against a subject's breast;
a breast milk collection portion that forms a cavity over the subject's areola when the device is attached to the subject's breast;
a sensor having an inlet and an outlet, the inlet being fluidly connected to the breast milk collection portion;
a faux nipple fluidly connected to the sensor outlet;
an attachment portion that fully or partially circumscribes the nipple and removeably adheres the device to the breast;
a memory to store a plurality of data elements measured by the sensor; and
a processor to execute instructions in the memory to:
store, in the memory, the data elements, and
send the data elements to a user device for graphical display

10. A method, comprising:
collecting breast milk from a subject's nipple;
measuring any combination of the flow rate, caloric content, or volume of breast milk during collection;
monitoring the flow rate, caloric content, and volume of breast milk ingested by an infant; and
displaying the measured data to a user device.
